(19) United States
(12) Patent Application Publication
(10) Pub. No.: US 2012/0152265 A1
(43) Pub. Date: Jun. 21, 2012

(54) Tobacco-Derived Syrup Composition

(75) Inventors: Michael Francis Dube, Winston-Salem, NC (US); William Monroe Coleman, III, Winston-Salem, NC (US); Anthony Richard Gerardi, Winston-Salem, NC (US)

(73) Assignee: R.J. Reynolds Tobacco Company

(21) Appl. No.: 12/971,746

(22) Filed: Dec. 17, 2010

(51) Int. Cl. C07D 401/04; A24B 15/10

(52) U.S. Cl. 131/352; 546/279.4

(57) ABSTRACT

The invention provides a tobacco composition for use in a smoking article or a smokeless tobacco composition that comprises a syrup derived from the stalk of a plant of the Nicotiana species. The invention also provides smoking articles and smokeless tobacco compositions that include the syrups described herein, and methods for preparing syrups derived from the stalk of a plant of the Nicotiana species for addition to a tobacco composition.

### FIELD OF THE INVENTION

The present invention relates to products made or derived from tobacco, or that otherwise incorporate tobacco, and are intended for human consumption.

### BACKGROUND OF THE INVENTION

Popular smoking articles, such as cigarettes, have a substantially cylindrical rod shaped structure and include a charge, roll or column of smokable material such as shredded tobacco (e.g., in cut filler form) surrounded by a paper wrapper thereby forming a so-called “tobacco rod.” Normally, a cigarette has a cylindrical filter element aligned in an end-to-end relationship with the tobacco rod. Typically, a filter element comprises plasticized cellulose acetate tow circumscribed by a paper material known as “plug wrap.” Certain cigarettes incorporate a filter element having multiple segments, and one of those segments can comprise activated charcoal particles. Typically, the filter element is attached to one end of the tobacco rod using a circumscribing wrapping material known as “tipping paper.” It also has become desirable to perforate the tipping material and plug wrap, in order to provide dilution of drawn mainstream smoke with ambient air. A cigarette is employed by a smoker by lighting one end thereof and burning the tobacco rod. The smoker then receives mainstream smoke into his/her mouth by drawing on the opposite end (e.g., the filter end) of the cigarette.

The tobacco used for cigarette manufacture is typically used in blended form. For example, certain popular tobacco blends, commonly referred to as “American blends,” comprise mixtures of flue-cured tobacco, burley tobacco and Oriental tobacco, and in many cases, certain processed tobaccos, such as reconstituted tobacco and processed tobacco stems. The precise amount of each type of tobacco within a tobacco blend used for the manufacture of a particular cigarette brand varies from brand to brand. However, for many tobacco blends, flue-cured tobacco makes up a relatively large proportion of the blend, while Oriental tobacco makes up a relatively small proportion of the blend. See, for example, Tobacco Encyclopedia, Voges (Ed.) p. 44-45 (1984), Browne, The Design of Cigarettes, 3^{rd} Ed., p. 43 (1990) and Tobacco Production, Chemistry and Technology, Davis et al. (Eds.) p. 346 (1999).

Tobacco also may be enjoyed in a so-called “smokeless” form. Particularly popular smokeless tobacco products are employed by inserting some form of processed tobacco or tobacco-containing formulation into the mouth of the user. See for example, the types of smokeless tobacco formulations, ingredients, and processing methodologies set forth in U.S. Pat. Nos. 1,376,586 to Schwartz; 3,696,917 to Levi; 4,513,756 to Pittman et al.; 4,528,993 to Sensabaugh, Jr. et al.; 4,624,269 to Story et al.; 4,991,599 to Tibbetts; 4,987,907 to Townsend; 5,092,352 to Sprinkle, III et al.; 5,387,416 to White et al.; 6,668,839 to Williams; 6,834,654 to Williams; 6,953,040 to Atchley et al.; 7,032,601 to Atchley et al.; and 7,694,686 to Atchley et al.; US Pat. Pub. Nos. 2004/0020503 to Williams; 2005/0115580 to Quinter et al.; 2005/0244521 to Strickland et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0029117 to Mua et al.; 2008/0173317 to Robinson et al.; 2008/0196730 to Engstrom et al.; 2008/0209586 to Neilsen et al.; 2008/0305216 to Crawford et al.; 2009/0065013 to Essen et al.; 2009/0293889 to Kumar et al.; and 2010/0291245 to Gao et al; PCT WO 04/095959 to Arnarp et al. and WO 2010/132444 A2 to Atchley; and U.S. patent application Ser. No. 12/638,394, filed Dec. 15, 2009, to Mua et al.; each of which is incorporated herein by reference.

Through the years, various treatment methods and additives have been proposed for altering the overall character or nature of tobacco materials utilized in tobacco products. For example, additives or treatment processes have been utilized in order to alter the chemistry or sensory properties of the tobacco material, or in the case of smokable tobacco materials, to alter the chemistry or sensory properties of mainstream smoke generated by smoking articles including the tobacco material. The sensory attributes of cigarette smoke can be enhanced by incorporating flavoring materials into various components of a cigarette. Exemplary flavoring additives include menthol and products of Maillard reactions, such as pyrazines, aminosugars, and Amadori compounds. American cigarette tobacco blends typically contain a casing composition that includes flavoring ingredients, such as licorice or cocoa powder and a sugar source such as high fructose corn syrup. See also, Leffingwell et al., Tobacco Flavoring for Smoking Products, R.J. Reynolds Tobacco Company (1972), which is incorporated herein by reference. Various processes for preparing flavorful and aromatic compositions for use in tobacco compositions are set forth in U.S. Pat. Nos. 3,424,171 to Rooker; 3,476,118 to Luttich; 4,150,677 to Osborne, Jr. et al.; 4,986,286 to Roberts et al.; 5,074,319 to White et al.; 5,099,862 to White et al.; 5,235,992 to Sensabaugh, Jr.; 5,301,694 to Raymond et al.; 6,298,858 to Coleman, III et al.; 6,325,860 to Coleman, III et al.; 6,428,624 to Coleman, III et al.; 6,440,223 to Dube et al.; 6,499,489 to Coleman, III; and 6,591,841 to White et al.; US Pat. Appl. Pub. Nos. 2004/0173228 to Coleman, III and 2010/0037903 to Coleman, III et al., each of which is incorporated herein by reference.

The sensory attributes of smokeless tobacco can also be enhanced by incorporation of certain flavoring materials. See, for example, US Pat. Appl. Pub. Nos. 2002/0162562 to Williams; 2002/0162563 to Williams; 2003/0070687 to Atchley et al.; 2004/0020503 to Williams, 2005/0178398 to Breslin et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0029117 to Mua et al.; 2008/0173317 to Robinson et al.; and 2008/0209586 to Neilsen et al., each of which is incorporated herein by reference.

It would be desirable to provide additional compositions and methods for altering the character and nature of tobacco (and tobacco compositions and formulations) useful in the manufacture of smoking articles and/or smokeless tobacco products. Specifically, it would be desirable to develop compositions and methods for altering the character and nature of tobacco compositions and formulations using tobacco-derived flavorful materials.

### SUMMARY OF THE INVENTION

The present invention provides a flavorful composition isolated from the Nicotiana species (i.e., a tobacco-derived composition) useful for incorporation into tobacco compositions utilized in a variety of tobacco products, such as smoking articles and smokeless tobacco products. The invention also provides methods for isolating components from the Nicotiana species (e.g., tobacco materials), and methods for processing those components and tobacco materials incorporating those components. In particular, the invention provides a tobacco-derived sugar-containing syrup that can be used as a flavorful tobacco composition and a method for forming such a syrup composition. The tobacco-derived syrup can be prepared by subjecting at least a portion of a tobacco plant (e.g., leaves, stalks, roots, or stems) to a process whereby liquid is extracted and concentrated in order to isolate desired flavorful components of the tobacco material.

In one aspect, the invention provides a flavorful tobacco composition for use in a tobacco product in the form of a sugar-containing syrup derived from the stalk of a plant of the Nicotiana species. In certain embodiments, the sugar-containing syrup is suitable for inclusion within a casing formulation or a top dressing formulation adapted for application to a tobacco material.

The sugar-containing syrup typically comprises sucrose, fructose, and glucose. In some embodiments, the sugar-containing syrup comprises at least about 50% by weight water and sugar combined or at least about 60% by weight water and sugar combined. In some embodiments, the sugar-containing syrup comprises at least about 15% by weight sugar compounds or at least about 20% by weight sugar compounds. For example, the sugar-containing syrup may comprise about 20% to about 60% by weight water and about 15% to about 40% by weight of sugar compounds, based on the total weight of the syrup composition. The sugar-containing syrup can have a specific gravity in the range of about 20 to about 50 g/cm³.

In another aspect, the present invention provides a tobacco product comprising a flavorful tobacco composition in the form of a sugar-containing syrup derived from the stalk of a plant of the Nicotiana species. In certain embodiments, the tobacco product further comprises a tobacco material or a non-tobacco plant material as a carrier for the sugar-containing syrup.

The tobacco product may be, for example, in the form of a smokeless tobacco composition. Exemplary smokeless tobacco compositions include moist snuff, dry snuff, chewing tobacco, tobacco-containing gums, and dissolvable or meltable tobacco products. The tobacco product may be, for example, in the form of a smoking article. In certain embodiments, the smoking article comprises a casing formulation or a top dressing comprising the sugar-containing syrup. The tobacco product may be, for example, in the form of an aerosol-generating device configured for non-combustion of plant material.

In yet another aspect, the invention provides a method for preparing a sugar-containing syrup from the stalk of a plant of the Nicotiana species, comprising: i) removing an aqueous liquid component comprising sugar compounds from the stalk of a plant of the Nicotiana species or a portion thereof; and ii) concentrating the aqueous liquid component to increase the specific gravity of the aqueous liquid component, which results in formation of a sugar-containing syrup suitable for use as a flavorful tobacco composition in a tobacco product.

The removing step may comprise, for example, pressing the aqueous liquid component from the stalk or contacting the stalk of the plant or portion thereof with a liquid to draw out the aqueous liquid component. The concentrating step may comprise, for example, heating the aqueous liquid component at atmospheric pressure. In certain embodiments, the concentrating step comprises concentrating the aqueous liquid component to a specific gravity of about 20 to about 50 g/cm³.

The method may further comprise filtering the aqueous liquid component to remove solid components, typically prior to the concentrating step. In some embodiments, one or more filter aids are used. In some embodiments, the aqueous liquid component is filtered during the concentrating step by skimming the solid components off the surface. The method may further comprise clarifying the aqueous liquid component by adding one or more clarifying agents to the aqueous liquid component.

The method for preparing a sugar-containing syrup may further comprise adding the sugar-containing syrup to a tobacco material or a non-tobacco plant material as a carrier for the sugar-containing syrup. Still further, the method may comprise incorporating the tobacco material or non-tobacco plant material into a tobacco product.

The tobacco product into which the tobacco material or non-tobacco plant material is incorporated may be, for example, in the form of a smokeless tobacco composition. Exemplary smokeless tobacco compositions include moist snuff, dry snuff, chewing tobacco, tobacco-containing gums, and dissolvable or meltable tobacco products. The tobacco product into which the tobacco material or non-tobacco plant material is incorporated may be, for example, in the form of a smoking article. In certain embodiments, the tobacco material or non-tobacco plant material comprises a casing formulation or top dressing comprising the sugar-containing syrup.

### BRIEF DESCRIPTION OF THE DRAWINGS

In order to provide an understanding of embodiments of the invention, reference is made to the appended drawings, which are not necessarily drawn to scale, and in which reference numerals refer to components of exemplary embodiments of the invention. The drawings are exemplary only, and should not be construed as limiting the invention.

FIG. 1 is an exploded perspective view of a smoking article having the form of a cigarette, showing the smokable material, the wrapping material components, and the filter element of the cigarette; and

FIG. 2 is a cross-sectional view of a smokeless tobacco product embodiment, taken across the width of the product, showing an outer pouch filled with a smokeless tobacco composition of the invention.

### DETAILED DESCRIPTION OF THE PREFERRED EMBODIMENTS

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water).

The present invention provides a concentrated form of extracted liquid from a plant from the Nicotiana species. The concentrated liquid provides a tobacco-derived sugar-containing syrup that can be used as a flavorful tobacco composition capable of replacing other sugar sources (e.g., high fructose corn syrup or honey) in tobacco products. As used herein, a “tobacco-derived syrup” refers to an aqueous liquid containing dissolved sugar compounds or other carbohydrates derived from a plant of the Nicotiana species. The syrup is typically used in a highly viscous form and typically contains water and sugar compounds as the primary ingredients. The terms “sugar” or “sugar compounds” refer to monosaccharides or polysaccharides (particularly short chain polysaccharides such as disaccharides or trisaccharides) characterized by a sweet taste, such as fructose, glucose, and sucrose. Sugar compounds are typically crystalline and water-soluble.

While the syrup of the invention can be derived from any portion of the tobacco plant (e.g., roots, stalks, leaves, flowers, and the like), the tobacco stalk is advantageous for use in the invention because the stalk contains a significant percentage of the water and sugar compounds present in tobacco, particularly in the central pith section of the tobacco stalk.

Tobacco plant stalks are typically considered to be a waste product, and are often discarded upon harvesting the tobacco plant. Tobacco plant stalks may comprise a number of components. For example, analysis of green tobacco stalks has revealed the presence of cellulose, hemi-cellulose, lignin, pectin, and sugars. The types and amount of sugar in a tobacco stalk may vary. Three dominant sugars typically found in green tobacco stalks are fructose, glucose, and sucrose. Tobacco stalks also typically comprise a significant amount of water. For example, the water content of a tobacco stalk may range from about 25% to about 90%, about 50% to about 80%, or about 60% to about 80% by weight.

According to the present invention, the tobacco stalks are harvested and then processed to remove an aqueous liquid component. Entire stalks or only portions of the stalks may be used in the process. The stalks may be directly processed in harvested form or may be physically altered by shredding or chopping prior to extraction of the liquid. As an alternative, the tobacco stalks may be prepared using a method by which the fibrous structure of the stalk rind surrounding the pith is kept intact. Such a method may allow the rind fibers to be used for another purpose. For example, a splitter device can be used to separate the rind from the pith. See, for example, U.S. Pat. Nos. 3,424,611, 3,424,612, and 3,464,877 to Miller et al.; 4,151,004 to Vukelic; and 3,976,498, 4,312,677 and 6,567,510 to Tilby et al., each of which is incorporated herein by reference. These references describe splitting methods to separate sugar cane into its individual parts, which may be adapted for use with tobacco stalks in accordance with the present invention. Thus, in some embodiments, only the separated pith (rather than tobacco stalk itself) is processed to provide a liquid component. It is to be understood that reference to processing “stalks” is also intended to encompass processing portions of stalks, e.g., separated pith components.

The stalks may be processed by any means to produce a liquid component. Two methods by which an aqueous liquid may be extracted from a tobacco plant material are pressure-based milling methods (i.e., applying pressure to press the liquid from the material) and liquid-based diffusion methods (i.e., passing water or other diluents through the material to carry off the liquid). These methods may be used independently or may be used in combination with one another.

With milling processes, the liquid from Nicotiana plant stalks is obtained by squeezing the liquid out of the tobacco stalks. The stalks can be, for example, crushed or pressed to extract the liquid component. For example, the stalks may be passed through one or more rollers (e.g., adjustable grooved rollers). The pressure exerted on the stalks may be varied. The stalks may be moistened prior to being crushed or pressed. Various milling methods and apparatus for the extraction of liquid from sugar cane are known, and may be adapted for use herein to extract a liquid component from tobacco stalks. See generally Dhavalikar, Manual for Sugar Cane Milling (2008), incorporated herein by reference. Specific milling techniques and equipment are discussed, for example, in U.S. Pat. Nos. 1,763,855 to Maxwell; 3,086,452 to French; 3,432,344 to Farmer; 3,969,802 to Bouvet; 4,077,316 to Georget; 4,925,115 to Pole et al.; and 5,855,168 to Nikam, each of which is incorporated herein by reference. Numerous other mill configurations for the pressing and/or crushing of sugar cane to obtain liquid therefrom are known and can be adapted for use in the methods described herein.

With diffusion methods, the tobacco plant stalks are contacted with a liquid to draw out the liquid component. Typically, the stalks are first prepared by rupturing some of the cells therein, such as by shredding or otherwise disintegrating or comminuting the stalks. The prepared stalks are then washed, typically repeatedly, with a treating liquid to extract the liquid contained within the tobacco plant stalks. The treating liquid may be any liquid in which one or more of the components to be extracted from the tobacco stalk is soluble. An exemplary treating liquid is water, particularly water at elevated temperature (e.g., water heated to a temperature of about 35° C. to about 75° C.). The treating liquid may be cycled and reused, such that dilute liquid extract from the tobacco stalk is used as the liquid to extract more liquid from the tobacco plant stalks. In some embodiments, one or more additives may be included in the treating liquid to afford more efficient extraction from the tobacco stalks. Although not wishing to be bound by theory, it is believed that the liquid component of the tobacco stalk is extracted in this method partly based on the physical action of repeated washings and draining of the liquid, and partly due to osmosis. Various types of diffusers are known and used for the extraction of liquid from sugar cane, which may be adapted for use with the tobacco stalks described herein. For example, sugar cane diffusers may extract a liquid component from the prepared sugar cane (a so-called “cane diffuser”) or from prepared sugar cane that has previously been milled (a so-called “bagasse diffuser”). Exemplary diffusion systems for the extraction of liquid from sugar cane (which may be adapted for use with tobacco stalks) are discussed, for example, in U.S. Pat. Nos. 4,182,632 to Cargill; 5,885,539 to Tosio; and 6,193,805 to Cargill, which are incorporated herein by reference. Numerous other diffusion methods and apparatus for such diffusion methods are known and can be adapted for use in the methods described herein.

As noted above, milling and diffusion methods may, in some embodiments, be combined to effectuate the isolation of liquid from tobacco stalks. For example, the tobacco stalks may be contacted with hot water following milling. See, for example, U.S. Pat. Nos. 3,661,082 to French et al.; 4,378,253 to Bouvet, and 5,073,200 to Leibig, which are incorporated herein by reference, which describe combined water maceration and milling processes for the extraction of sucrose from sugar cane, the teachings of which may be adapted for use with tobacco stalks according to the present invention. Hot water maceration may, in some cases, enable the extraction of more liquid and/or more material from the tobacco stalk than is achievable with milling methods alone. The hot water can penetrate into cells that are ruptured by milling and effectively leach or flush out additional encapsulated liquid from the cells.

The raw, extracted liquid obtained from the tobacco stalks using the techniques noted above is collected and optionally subjected to one or more clarification and/or filtration steps. Where both clarification and filtration steps are implemented, such steps can be performed in any order (i.e., the liquid may be first clarified and then filtered or filtered and then clarified). Multiple clarification and filtration steps may be performed in combination (e.g., the liquid may be filtered, clarified, and filtered again). Further, clarification, filtration or both may be performed at later stages of the process, such as following concentration to form the syrup as described below. Although these methods are described herein as related to the raw, extracted liquid, these process steps are applicable at any stage of the syrup preparation process.

Clarification typically involves the addition of various materials (denoted clarifying agents) to the raw, extracted liquid. For example, specific clarifying agents that may be added to the raw, extracted liquid include, but are not limited to, various salts, lime, sulfur, and other compounds to adjust the pH or otherwise stabilize or clarify the liquid. In some embodiments, the clarifying agents also function as flocculants, which can facilitate the removal of one or more impurities. For example, flocculants may remove suspended particles and/or dissolved molecules or ions. The flocculant can be removed from the liquid by any means (e.g., filtration, settling, centrifugation, etc.). The liquid may be heated to initiate the desired reactions for clarification. In certain embodiments, the extracted liquid is heated at a temperature sufficiently low enough to avoid excessive inversion of sucrose (e.g., less than about 76° C.). For examples of materials and methods that have been used for the clarification of liquid extracted from sugar cane, see U.S. Pat. Nos. 3,418,165 to Rabe, 3,746,265 to Gil et al.; 3,808,050 to Paley; 3,994,743 to Paley; 4,968,353 to Kawasaki et al.; 5,262,328 to Clarke et al.; and 6,245,153 to Gonzales, each of which is incorporated by reference herein. These examples as well as others that would be apparent to one of skill in the art for the clarification of sugar-containing liquids may be applied and/or adapted for use in the presently disclosed method.

The raw, extracted liquid and/or the clarified liquid will typically contain some percentage of solid components. Thus, in certain embodiments, the extracted liquid component is filtered to remove solids. The process of filtration can comprise passing the liquid through one or more filter screens to remove selected sizes of particulate matter. Screens may be, for example, stationary, vibrating, rotary, or any combination thereof. Filters may be, for example, press filters or pressure filters. In some embodiments, the filtration method used may involve microfiltration, ultrafiltration, or nanofiltration. A filter aid may be employed to provide effective filtration and may comprise any material typically used for this purpose. For example, some common filter aids include cellulose fibers, perlite, bentonite, diatomaceous earth and other silaceous materials. To remove solid components, alternative methods may also be used, for example, centrifugation or settling/sedimentation of the components and siphoning off of the liquid. See, for example, U.S. Pat. Nos. 5,468,300 and 5,468,301 to Monclin, both of which are incorporated by reference herein.

In one embodiment, the process of the invention involves processing the extracted liquid using an ultrafiltration technique. In ultrafiltration processing, the extracted liquid is exposed to a membrane having a pore size capable of excluding small molecular weight components, typically in a cross-flow arrangement. The pore size of membranes typically utilized in ultrafiltration can vary, but generally falls within the range of about 0.1 to about 0.001 micron. Ultrafiltration membranes can also be characterized by their nominal molecular weight limit (NMWL), which is an approximation of the upper limit of the molecular weight of species capable of passing through the membrane. For purposes of the present invention, the NMWL is typically between about 5,000 Da and about 75,000 Da. In one embodiment, the ultrafiltration process involves passing the extracted liquid through multiple ultrafiltration stages having different NMWL ratings. For example, the process could involve first processing the extracted liquid using a 50,000 Da ultrafiltration membrane and thereafter processing the liquid using a 5,000 Da ultrafiltration membrane. Although various types of ultrafiltration membranes can be used, a cellulose-based hollow fiber membrane is one advantageous choice. Such membranes are commercially available from Koch Membrane Systems, Inc. Use of ultrafiltration techniques are set forth, for example, in U.S. Pat. No. 4,941,484 to Clapp et al, which is incorporated by reference herein.

Following extraction, filtration and/or clarification, the liquid can be further processed if desired. For example, the liquid can be processed in a manner adapted to concentrate the dissolved or dispersed components of the liquid by removing at least a portion of the solvent (i.e., water). The concentration step removes water from the extracted aqueous liquid, which results in a syrup having an increased concentration of sugar compounds.

Various methods of solvent removal can be used, such as heat treatment to evaporate the solvent, reverse osmosis membrane treatment, spray drying or freeze drying. In one embodiment, the concentration process can entail heating the extracted liquid in a vented vessel to evaporate a portion of the water. The temperature and pressure at which the liquid is heated may vary. For example, an aqueous liquid may be boiled at atmospheric pressure at or above about 100° C. For example, the filtered, extracted liquid may be boiled in an open vessel (e.g., a kiln) to produce the syrup. In certain embodiments, the liquid may be heated at a pressure other than atmospheric, such as under a partial vacuum (thereby reducing the temperature required to boil the aqueous liquid) or at increased pressure above atmospheric pressure (thereby increasing the temperature required to boil the aqueous liquid).

In one embodiment, a multiple-effect evaporator may be used to concentrate the liquid into a syrup. The multiple-effect evaporator may have any number of effects (e.g., up to about 8). A multiple-effect evaporator is an evaporation system that connects a series of evaporation bodies and uses the thermal energy contained in the vapor and the pressure and temperature differences between the evaporation bodies to concentrate the liquid in a more efficient manner.

During the conversion of the extracted liquid into a syrup, solids may be produced. For example, proteins may solidify and rise to the surface of the liquid during the concentration process. Accordingly, it may be necessary to skim the mixture at one or more points during the process of heating or otherwise concentrating the liquid. Skimming the mixture can be done, for example, using thin screens.

The liquid may be concentrated to give a syrup having a predetermined thickness or density. For example, the syrup may be concentrated to achieve a specific gravity range of about 20 to about 50 g/cm³, preferably about 30 to about 36 g/cm³. The desired thickness can be achieved, for example, by continually monitoring the specific gravity of the liquid and removing the heat and/or vacuum when the desired specific gravity is achieved. Specific gravity may be analyzed by any means, for example, using a hydrometer. Alternatively, in some embodiments, a refractometer may be used. Because specific gravity is temperature-dependent, in some cases, the specific gravity values obtained may need to be corrected to obtain accurate readings for liquids at different temperatures.

Following concentration, the resulting syrup may be used directly or may be further processed. For example, additional clarification and/or filtration steps may be performed. In certain embodiments, the syrup may be decolorized and/or de-ashed. In addition, if desired, the syrup can be subjected to separation processes adapted to separate the various sugar compounds into isolated fractions. For example, chromatographic techniques could be used to separate a fructose-enriched fraction from a sucrose-enriched fraction.

The yield of syrup from tobacco stalks may vary. Yield is dependent on a number of factors. For example, yield may depend on the quality of the tobacco stalk. Poor quality stalks or stalks that have been harvested very early or very late may comprise different amounts of liquid components and/or may comprise liquid with varying levels of sugars. Yield may also depend on the efficiency of liquid extraction. The efficiency of liquid extraction is somewhat controlled by the extraction method and the specific equipment used. Yield may also vary as a result of the specific conditions used throughout the syrup production process. For example, yield may be improved by careful control of the boiling process, such as by completing the boiling process quickly.

The exact composition of the resulting syrup may vary. However, it typically comprises a significant amount of sugar compounds and water. In certain embodiments, the syrup comprises at least about 50% by weight water and sugar combined, at least about 60% by weight water and sugar combined, or at least about 70% by weight water and sugar combined, based on the total weight of the syrup composition. The predominant sugar compounds present in the syrup typically include sucrose, glucose, and fructose. The syrup typically comprises at least about 15% by weight sugar compounds, or at least about 20% by weight sugar compounds, or at least about 25% by weight sugar compounds, based on the total weight of the syrup composition. The syrup may also contain other flavorful compounds such as pyrazines resulting from Maillard reactions between the sugar compounds and nitrogen sources in the liquid, thermal degradation products derived from the sugar compounds (e.g., furans), and other flavorants such as damascone, norsolanadione, solanone, and ionone derivatives. In one embodiment, the tobacco-derived syrup of the invention contains about 20% to about 60% by weight water (e.g., about 30% to about 50% by weight) and about 15% to about 40% by weight (e.g., about 20% to about 40% by weight) of sugar compounds, based on the total weight of the syrup composition.

Control over the conditions of processing the tobacco stalks may allow some degree of control over the ratio of sucrose, glucose, and fructose. Sucrose present in the tobacco stalks may “decay” into glucose and fructose (i.e., undergo inversion) under certain conditions. For example, high temperatures and acidic conditions may promote inversion; accordingly, control over the temperature and/or pH of the liquid at various stages of the syrup production described herein can affect the ratio of sugars in the final syrup product. In other words, processing conditions involving lower temperatures and/or higher pH may lead to a syrup having a higher concentration of sucrose, whereas processing conditions involving higher temperatures and/or lower pH may lead to a syrup having a lower concentration of sucrose (and correspondingly, a higher concentration of glucose and/or fructose).

Following extraction of the liquid component, the remaining fibrous stalk material can also be incorporated into tobacco products. For instance, the fibrous material can be added to a tobacco blend for use in a smoking article or a smokeless tobacco composition as a filler or tobacco substitute. In one embodiment, the fibrous tobacco stalk material can serve as a carrier for a tobacco extract. See, for example, the manner in which fibrous materials are incorporated into tobacco products in U.S. Pat. Nos. 2,576,021 and 2,809,904, both to Koree, which are incorporated by reference herein.

The selection of the plant from the Nicotiana species utilized in the process of the invention can vary; and in particular, the types of tobacco or tobaccos may vary. The type of tobacco used as both the source of tobacco stalks and as the carrier for the syrup of the invention can vary. Tobaccos that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos. Descriptions of various types of tobaccos, growing practices and harvesting practices are set forth in Tobacco Production, Chemistry and Technology, Davis et al. (Eds.) (1999), which is incorporated herein by reference. Various representative types of plants from the Nicotiana species are set forth in Goodspeed, The Genus Nicotiana, (Chonica Botanica) (1954); U.S. Pat. Nos. 4,660,577 to Sensabaugh, Jr. et al.; 5,387,416 to White et al. and 7,025,066 to Lawson et al.; US Patent Appl. Pub. Nos. 2006/0037623 to Lawrence, Jr. and 2008/0245377 to Marshall et al.; each of which is incorporated herein by reference.

The particular Nicotiana species of material used in the invention could also vary. Of particular interest are N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, and N. x sanderae. Also of interest are N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. rustica, N. simulans, N. stocktonii, N. suaveolens, N. tabacum, N. umbratica, N. velutina, and N. wigandioides. Other plants from the Nicotiana species include N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia and N. spegazzinii. The Nicotiana species can be derived using genetic-modification or crossbreeding techniques (e.g., tobacco plants can be genetically engineered or crossbred to increase or decrease production of certain components or to otherwise change certain characteristics or attributes). See, for example, the types of genetic modifications of plants set forth in U.S. Pat. Nos. 5,539,093 to Fitzmaurice et al.; 5,668,295 to Wahab et al.; 5,705,624 to Fitzmaurice et al.; 5,844,119 to Weigl; 6,730,832 to Dominguez et al.; 7,173,170 to Liu et al.; 7,208,659 to Colliver et al.; and 7,230,160 to Benning et al.; US Patent Appl. Pub. No. 2006/0236434 to Conkling et al.; and PCT WO 2008/103935 to Nielsen et al.

For the preparation of smokeless and smokable tobacco products, it is typical for harvested plants of the Nicotiana species to be subjected to a curing process. Descriptions of various types of curing processes for various types of tobaccos are set forth in Tobacco Production, Chemistry and Technology, Davis et al. (Eds.) (1999). Exemplary techniques and conditions for curing flue-cured tobacco are set forth in Nestor et al., Beitrage Tabakforsch. Int., 20, 467-475 (2003) and U.S. Pat. No. 6,895,974 to Peele, which are incorporated herein by reference. Representative techniques and conditions for air curing tobacco are set forth in Roton et al., Beitrage Tabakforsch. Int., 21, 305-320 (2005) and Staaf et al., Beitrage Tabakforsch. Int, 21, 321-330 (2005), which are incorporated herein by reference. Certain types of tobaccos can be subjected to alternative types of curing processes, such as fire curing or sun curing. Preferably, harvested tobaccos are cured and then aged.

The stalk of the plant of the Nicotiana species can be employed in an immature form. That is, the plant can be harvested before the plant reaches a stage normally regarded as ripe or mature. As such, for example, the plant can be harvested when the tobacco plant is at the point of a sprout, is commencing leaf formation, is commencing flowering, or the like.

The stalk of the plant of the Nicotiana species can be employed in a mature form. That is, the plant can be harvested when that plant reaches a point that is traditionally viewed as being ripe, over-ripe or mature. As such, for example, through the use of tobacco harvesting techniques conventionally employed by farmers, Oriental tobacco plants can be harvested, burley tobacco plants can be harvested, or Virginia tobacco leaves can be harvested or primed by stalk position.

After harvest, the plant of the Nicotiana species, or portion thereof, can be used in a green form (e.g., tobacco can be used without being subjected to any curing process). For example, tobacco in green form can be frozen, freeze-dried, subjected to irradiation, yellowed, dried, cooked (e.g., roasted, fried or boiled), or otherwise subjected to storage or treatment for later use. Such tobacco also can be subjected to aging conditions.

Syrup generated according to the process of the invention is useful as a flavorful material for tobacco compositions, particularly tobacco compositions incorporated into smoking articles or smokeless tobacco products. In accordance with the present invention, a tobacco product incorporates tobacco that is combined with a tobacco-derived syrup according to the invention. That is, a portion of the tobacco product can be comprised of some form of syrup prepared according to the invention. Addition of the syrup to a tobacco composition can enhance a tobacco composition in a variety of ways, depending on the nature of the syrup generated and the type of tobacco composition. Exemplary syrup compositions can serve to provide flavor and/or aroma to a tobacco product (e.g., the composition can alter the sensory characteristics of tobacco compositions or smoke derived therefrom). Given the significant sugar content in the syrup of the invention, the syrup made according to the present invention can serve as a replacement for traditional sugar-enriched components of a tobacco product (e.g., corn syrup or honey) or as a replacement for certain sweeteners (e.g., natural sweeteners such as fructose, sucrose, glucose, maltose, vanillin, ethylvanillin glucoside, mannose, galactose, lactose, and the like). For example, the syrup may be utilized in the casing of a cigarette to add flavor typically derived from one or more of the traditional components of a cigarette casing, particularly the sugar-enriched components of a casing material such as high fructose corn syrup.

The syrup can be employed in a variety of forms. The syrup can be employed in a liquid form, and as such, the content of tobacco solubles within the liquid solvent can be controlled by concentration of the syrup by removal of solvent, addition of solvent to dilute the syrup, or the like. Alternatively, the tobacco-derived syrup can be isolated in an essentially solvent free form, such as can be obtained using a spray drying or freeze drying process.

The tobacco product to which the syrup of the invention is added can vary, and may include any product configured or adapted to deliver tobacco or some component thereof to the user of the product. Exemplary tobacco products include smoking articles (e.g., cigarettes), smokeless tobacco products, and aerosol-generating devices that contain a tobacco material or other plant material that is not combusted during use. The incorporation of the syrup of the invention into a tobacco product may involve use of a tobacco material or non-tobacco plant material as a carrier for the syrup, such as by absorbing the syrup into the tobacco or other plant material or otherwise associating the syrup with the carrier material. The types of tobacco that can serves as the carrier for the syrup of the invention can vary, and can include any of the tobacco types discussed herein, including various cured tobacco materials (e.g., flue-cured or air-cured tobaccos) or portions thereof (e.g., tobacco lamina or tobacco stems). The physical configuration of the tobacco material to which the syrup is added can also vary, and can include tobacco materials in shredded or particulate form, or in the form of a sheet (e.g., reconstituted tobacco sheets) or in whole leaf form.

In one embodiment, the syrup of the invention is used as a flavorful tobacco composition in the manufacture of smoking articles. For example, the syrup prepared in accordance with the present invention can be mixed with casing materials and applied to tobacco as a casing ingredient (e.g., using the types of methods set forth in U.S. Pat. No. 4,819,668 to Shelar, which is incorporated herein by reference), incorporated into smoking articles as a top dressing ingredient, or incorporated into reconstituted tobacco materials (e.g., using the types of tobacco reconstitution processes generally set forth in US Pat. Nos. 5,143,097 to Sohn; 5,159,942 to Brinkley et al.; 5,598,868 to Jakob; 5,715,844 to Young; 5,724,998 to Gellatly; and 6,216,706 to Kumar, which are incorporated herein by reference). Still further, the syrup of the invention can be incorporated into a cigarette filter (e.g., in the filter plug, plug wrap, or tipping paper) or incorporated into cigarette wrapping paper, preferably on the inside surface, during the cigarette manufacturing process.

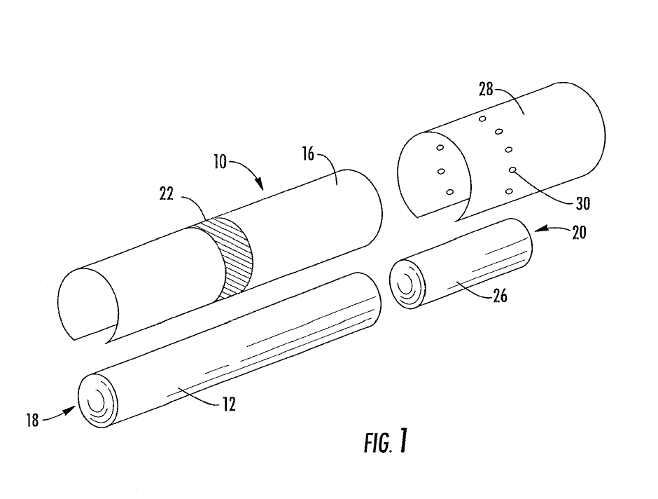

Referring to FIG. 1, there is shown a smoking article 10 in the form of a cigarette and possessing certain representative components of a smoking article that can contain the syrup of the present invention. The cigarette 10 includes a generally cylindrical rod 12 of a charge or roll of smokable filler material (e.g., about 0.3 to about 1.0 g of smokable filler material such as tobacco material) contained in a circumscribing wrapping material 16. The rod 12 is conventionally referred to as a “tobacco rod.” The ends of the tobacco rod 12 are open to expose the smokable filler material. The cigarette 10 is shown as having one optional band 22 (e.g., a printed coating including a film-forming agent, such as starch, ethylcellulose, or sodium alginate) applied to the wrapping material 16, and that band circumscribes the cigarette rod in a direction transverse to the longitudinal axis of the cigarette. The band 22 can be printed on the inner surface of the wrapping material (i.e., facing the smokable filler material), or less preferably, on the outer surface of the wrapping material.

At one end of the tobacco rod 12 is the lighting end 18, and at the mouth end 20 is positioned a filter element 26. The filter element 26 positioned adjacent one end of the tobacco rod 12 such that the filter element and tobacco rod are axially aligned in an end-to-end relationship, preferably abutting one another. Filter element 26 may have a generally cylindrical shape, and the diameter thereof may be essentially equal to the diameter of the tobacco rod. The ends of the filter element 26 permit the passage of air and smoke therethrough.

A ventilated or air diluted smoking article can be provided with an optional air dilution means, such as a series of perforations 30, each of which extend through the tipping material and plug wrap. The optional perforations 30 can be made by various techniques known to those of ordinary skill in the art, such as laser perforation techniques. Alternatively, so-called off-line air dilution techniques can be used (e.g., through the use of porous paper plug wrap and pre-perforated tipping paper).

The syrup of the invention can also be incorporated into aerosol-generating devices that contain tobacco material (or some portion or component thereof) that is not intended to be combusted during use. Exemplary references that describe smoking articles of a type that generate flavored vapor, visible aerosol, or a mixture of flavored vapor and visible aerosol, include U.S. Pat. Nos. 3,258,015 to Ellis et al.; 3,356,094 to Ellis et al.; 3,516,417 to Moses; 4,347,855 to Lanzellotti et al.; 4,340,072 to Bolt et al.; 4,391,285 to Burnett et al.; 4,917,121 to Riehl et al.; 4,924,886 to Litzinger; and 5,060,676 to Hearn et al., all of which are incorporated by reference herein. Many of these types of smoking articles employ a combustible fuel source that is burned to provide an aerosol and/or to heat an aerosol-forming material. See, for example, U.S. Pat. Nos. 4,756,318 to Clearman et al.; 4,714,082 to Banerjee et al.; 4,771,795 to White et al.; 4,793,365 to Sensabaugh et al.; 4,917,128 to Clearman et al.; 4,961,438 to Korte; 4,966,171 to Serrano et al.; 4,969,476 to Bale et al.; 4,991,606 to Serrano et al.; 5,020,548 to Farrier et al.; 5,033,483 to Clearman et al.; 5,040,551 to Schlatter et al.; 5,050,621 to Creighton et al.; 5,065,776 to Lawson; 5,076,296 to Nystrom et al.; 5,076,297 to Farrier et al.; 5,099,861 to Clearman et al.; 5,105,835 to Drewett et al.; 5,105,837 to Barnes et al.; 5,115,820 to Hauser et al.; 5,148,821 to Best et al.; 5,159,940 to Hayward et al.; 5,178,167 to Riggs et al.; 5,183,062 to Clearman et al.; 5,211,684 to Shannon et al.; 5,240,014 to Deevi et al.; 5,240,016 to Nichols et al.; 5,345,955 to Clearman et al.; 5,551,451 to Riggs et al.; 5,595,577 to Bensalem et al.; 5,819,751 to Barnes et al.; 6,089,857 to Matsuura et al.; 6,095,152 to Beven et al; 6,578,584 to Beven; and 6,730,832 to Dominguez; which are incorporated herein by reference. Furthermore, certain types of cigarettes that employ carbonaceous fuel elements have been commercially marketed under the brand names “Premier” and “Eclipse” by R. J. Reynolds Tobacco Company. See, for example, those types of cigarettes described in Chemical and Biological Studies on New Cigarette Prototypes that Heat Instead of Burn Tobacco, R. J. Reynolds Tobacco Company Monograph (1988) and Inhalation Toxicology, 12:5, p. 1-58 (2000). Addition types of aerosol-generating devices are described in U.S. Pat. No. 7,726,320 to Robinson et al. and US Pat. Appl. Pub. Nos. 2006/0196518 and 2007/0267031, both to Hon, all of which are incorporated by reference herein.

The syrup of the invention can be incorporated into smokeless tobacco products, such as loose moist snuff (e.g., snus), loose dry snuff, chewing tobacco, pelletized tobacco pieces (e.g., having the shapes of pills, tablets, spheres, coins, beads, obloids or beans), extruded or formed tobacco strips, pieces, rods, cylinders or sticks, finely divided ground powders, finely divided or milled agglomerates of powdered pieces and components, flake-like pieces, molded processed tobacco pieces, pieces of tobacco-containing gum, rolls of tape-like films, readily water-dissolvable or water-dispersible films or strips (e.g., US Pat. App. Pub. No. 2006/0198873 to Chan et al.), or capsule-like materials possessing an outer shell (e.g., a pliable or hard outer shell that can be clear, colorless, translucent or highly colored in nature) and an inner region possessing tobacco or tobacco flavor (e.g., a Newtonian fluid or a thixotropic fluid incorporating tobacco of some form). Various types of smokeless tobacco products are set forth in U.S. Pat. Nos. 1,376,586 to Schwartz; 3,696,917 to Levi; 4,513,756 to Pittman et al.; 4,528,993 to Sensabaugh, Jr. et al.; 4,624,269 to Story et al.; 4,987,907 to Townsend; 5,092,352 to Sprinkle, III et al.; and 5,387,416 to White et al.; US Pat. App. Pub. Nos. 2005/0244521 to Strickland et al. and 2008/0196730 to Engstrom et al.; PCT WO 04/095959 to Arnarp et al.; PCT WO 05/063060 to Atchley et al.; PCT WO 05/016036 to Bjorkholm; and PCT WO 05/041699 to Quinter et al., each of which is incorporated herein by reference. See also, the types of smokeless tobacco formulations, ingredients, and processing methodologies set forth in U.S. Pat. Nos. 6,953,040 to Atchley et al. and 7,032,601 to Atchley et al.; US Pat. Appl. Pub. Nos. 2002/0162562 to Williams; 2002/0162563 to Williams; 2003/0070687 to Atchley et al.; 2004/0020503 to Williams, 2005/0178398 to Breslin et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0029117 to Mua et al.; 2008/0173317 to Robinson et al.; 2008/0209586 to Neilsen et al.; 2010/0018541 to Gerardi et al.; 2010/0018540 to Doolittle et al.; and 2010/0116281 to Marshall et al., each of which is incorporated herein by reference.

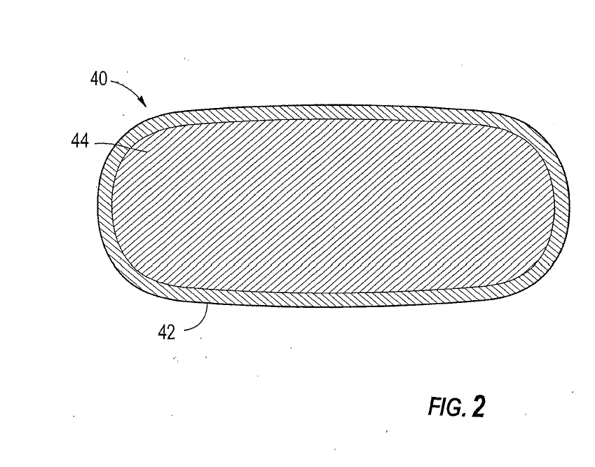

Referring to FIG. 2, a representative snus type of tobacco product comprising the syrup of the present invention is shown. In particular, FIG. 2 illustrates a smokeless tobacco product 40 having a water-permeable outer pouch 42 containing a smokeless tobacco composition 44, wherein the tobacco composition includes a shredded or particulate tobacco material serving as a carrier for the syrup of the invention.

Many exemplary smokeless tobacco compositions that can benefit from use of the syrup of the invention comprise shredded or particulate tobacco material that can serve as a carrier for the flavorful syrup of the invention. The smokeless tobacco compositions of the invention can also include a water-soluble polymeric binder material and optionally other ingredients that provide a dissolvable composition that will slowly disintegrate in the oral cavity during use. In certain embodiments, the smokeless tobacco composition can include lipid components that provide a meltable composition that melts (as opposed to merely dissolving) in the oral cavity, such as compositions set forth in U.S. Appl. No. 12/854,342 to Cantrell et al., filed Aug. 11, 2010, and which is incorporated by reference herein.

In one particular smokeless tobacco product embodiment, the syrup of the invention is added to a non-tobacco plant material, such as a plant material selected from potato, beet (e.g., sugar beet), grain, pea, apple, and the like. The non-tobacco plant material can be used in a processed form. In certain preferred embodiments, the non-tobacco plant material can be used in an extracted form, and as such, at least a portion of certain solvent soluble components are removed from that material. The non-tobacco extracted plant material is typically highly extracted, meaning a substantial amount of the aqueous soluble portion of the plant material has been removed. For example, a water-extracted pulp can be obtained by extracting significant amounts of water soluble components from the plant material. For example, certain water-extracted plant materials can comprise less than about 20 weight percent, and often less than about 10 weight percent water soluble components; and depending upon processing conditions, certain water-extracted plant materials can be virtually free of water soluble components (e.g., less than about 1 weight percent water soluble components). One preferred water-extracted plant material is water extracted sugar beet pulp (e.g., water extracted sugar beet leaf pulp). The extracted non-tobacco plant material is typically used in a form that can be described as shredded, ground, granulated, fine particulate, or powder form.

Further additives can be admixed with, or otherwise incorporated within, the smokeless tobacco compositions according to the invention. The additives can be artificial, or can be obtained or derived from herbal or biological sources. Exemplary types of additives include salts (e.g., sodium chloride, potassium chloride, sodium citrate, potassium citrate, sodium acetate, potassium acetate, and the like), natural sweeteners (e.g., fructose, sucrose, glucose, maltose, vanillin, ethylvanillin glucoside, mannose, galactose, lactose, and the like), artificial sweeteners (e.g., sucralose, saccharin, aspartame, acesulfame K, neotame and the like), organic and inorganic fillers (e.g., grains, processed grains, puffed grains, maltodextrin, dextrose, calcium carbonate, calcium phosphate, corn starch, lactose, manitol, xylitol, sorbitol, finely divided cellulose, and the like), binders (e.g., povidone, sodium carboxymethylcellulose and other modified cellulosic types of binders, sodium alginate, xanthan gum, starch-based binders, gum arabic, lecithin, and the like), pH adjusters or buffering agents (e.g., metal hydroxides, preferably alkali metal hydroxides such as sodium hydroxide and potassium hydroxide, and other alkali metal buffers such as metal carbonates, preferably potassium carbonate or sodium carbonate, or metal bicarbonates such as sodium bicarbonate, and the like), colorants (e.g., dyes and pigments, including caramel coloring and titanium dioxide, and the like), humectants (e.g., glycerin, propylene glycol, and the like), oral care additives (e.g., thyme oil, eucalyptus oil, and zinc), preservatives (e.g., potassium sorbate, and the like), syrups (e.g., honey, high fructose corn syrup, and the like), disintegration aids (e.g., microcrystalline cellulose, croscarmellose sodium, crospovidone, sodium starch glycolate, pregelatinized corn starch, and the like), flavorant and flavoring mixtures, antioxidants, and mixtures thereof. If desired, the additive can be microencapsulated as set forth in US Patent Appl. Pub. No. 2008/0029110 to Dube et al., which is incorporated by reference herein. In addition, exemplary encapsulated additives are described, for example, in WO 2010/132444 A2 to Atchley, which has been previously incorporated by reference herein.

The amount of syrup incorporated within a tobacco composition or tobacco product can depend on the desired function of the syrup, the chemical makeup of the syrup, and the type of tobacco composition to which the syrup is added. The amount of syrup added to a tobacco composition can vary, but will typically not exceed about 5 weight percent based on the total dry weight of the tobacco composition to which the syrup is added. For example, the amount of syrup added to a tobacco composition may be in the range of about 0.25 to about 5 weight percent based on the total dry weight of the tobacco composition. In a specific embodiment, the syrup is added to a tobacco composition comprising a blend of tobaccos including burley tobacco and may be in the range of about 5.5% to about 19% by weight of the burley portion of the blend.

### EXPERIMENTAL

Aspects of the present invention is more fully illustrated by the following examples, which are set forth to illustrate certain aspects of the present invention and are not to be construed as limiting thereof.

### Example 1

### Preparation of Syrup from Tobacco Stalks

Flue-cured tobacco stalks (˜4000 pounds) are harvested. The stalks have been in the field for approximately 5 weeks post priming. The stalks are manually fed through a press designed to squeeze liquid from sugar cane stalks (adjusted slightly to allow stalks to pass through). About 70 gallons (560 pounds) of liquid are collected. The liquid is transferred to a kiln. The fines suspended in the liquid settle out on the bottom of the kiln. The liquid is heated in the kiln and begins to boil in about 30 minutes. A greenish foamy layer forms on top and is skimmed off and discarded. After 30 minutes of boiling, the density of the liquid appears to be roughly that of water. The color has changed from green to brown/yellow.

After another 60 minutes of boiling, the density of the liquid is about 12 g/cm³. After another 30 minutes, the density of the liquid is about 30 g/cm³ and heat is removed. The overall yield of syrup is about 3 gallons. Upon cooling, a precipitate begins to form within about 15 minutes, which settles to the bottom.

### Example 2

### Chemical Analysis of Syrup

To three 125 mL separatory funnels are added 20 g of tobacco stalk syrup from Example 1. To one funnel is added 50 mL of methylene chloride, to one funnel is added 50 mL of hexane, and to one funnel is added 50 mL of methyl t-butyl ether. All three flasks are shaken vigorously and allowed to stand overnight. The solvents are drawn off, dried over anhydrous sodium sulfate, and concentrated to about 2 mL using a rotary evaporator. The resulting samples are analyzed by gas chromatography (e.g., Agilent 6890 GC).

The gas chromatograms show that the syrup contains compounds associated with sugar nitrogen reactions, such as dimethyl pyrazine. The syrup also contains sugar thermal degradation reaction products such as furan derivatives. Still further, there is evidence of several additional flavor materials such as damascone, norsolanadione, solanone, and ionone derivatives.

Non-volatile components containing a functional group like OH can be derivatized to form a volatile silane derivative. For example, non-volatile sugars such as fructose, glucose, and sucrose can be rendered volatile when converted to their silane derivatives. Derivatization is performed on the syrup and the components are identified by gas chromatography and their relative percent values are calculated.

The data shows that the major components detected are sugars (fructose, glucose, sucrose, and other sugars). The percent contribution from the sugars to the total area is 77%. The total of the three sugars is 282.2 gm/ml, or 28% by weight in the syrup. Water represents 40% by weight of the total syrup. Thus, water and sugar make up almost 70% of the syrup. Nicotine content is found to be very low (0.5%) in the syrup.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed:

1. A flavorful tobacco composition for use in a tobacco product in the form of a sugar-containing syrup derived from the stalk of a plant of the Nicotiana species.

2. The tobacco composition of claim 1, wherein the sugar-containing syrup is contained within a casing formulation or a top dressing formulation adapted for application to a tobacco material.

3. The tobacco composition of claim 1, wherein the sugar-containing syrup comprises sucrose, fructose, and glucose.

4. The tobacco composition of claim 1, wherein the sugar-containing syrup comprises at least about 50% by weight water and sugar combined.

5. The tobacco composition of claim 4, wherein the sugar-containing syrup comprises at least about 60% by weight water and sugar combined.

6. The tobacco composition of claim 1, wherein the sugar-containing syrup comprises at least about 15% by weight sugar compounds.

7. The tobacco composition of claim 1, wherein the sugar-containing syrup comprises at least about 20% by weight sugar compounds.

8. The tobacco composition of claim 1, wherein the sugar-containing syrup comprises about 20% to about 60% by weight water and about 15% to about 40% by weight of sugar compounds, based on the total weight of the syrup composition.

9. The tobacco composition of claim 1, wherein the sugar-containing syrup has a specific gravity in the range of about 20 to about 50 g/cm³.

10. A tobacco product comprising a flavorful tobacco composition in the form of a sugar-containing syrup derived from the stalk of a plant of the Nicotiana species.

11. The tobacco product of claim 10, further comprising a tobacco material or a non-tobacco plant material as a carrier for the sugar-containing syrup.

12. The tobacco product of claim 10, wherein the tobacco product is in the form of a smokeless tobacco composition.

13. The tobacco product of claim 12, wherein the form of the smokeless tobacco composition is selected from the group consisting of moist snuff, dry snuff, chewing tobacco, tobacco-containing gums, and dissolvable or meltable tobacco products.

14. The tobacco product of claim 10, wherein the tobacco product is in the form of a smoking article.

15. The tobacco product of claim 14, wherein the smoking article comprises a casing formulation or a top dressing comprising the sugar-containing syrup.

16. The tobacco product of claim 10, wherein the tobacco product is in the form of an aerosol-generating device configured for non-combustion of plant material.

17. The tobacco product of claim 10, wherein the sugar-containing syrup comprises at least about 50% by weight water and sugar combined.

18. The tobacco product of claim 17, wherein the sugar-containing syrup comprises at least about 60% by weight water and sugar combined.

19. The tobacco product of claim 10, wherein the sugar-containing syrup comprises at least about 15% by weight sugar compounds.

20. The tobacco product of claim 10, wherein the sugar-containing syrup comprises at least about 20% by weight sugar compounds.

21. The tobacco product of claim 10, wherein the sugar-containing syrup comprises about 20% to about 60% by weight water and about 15% to about 40% by weight of sugar compounds, based on the total weight of the syrup composition.

22. The tobacco product of claim 10, wherein the sugar-containing syrup has a specific gravity in the range of about 20 to about 50 g/cm³.

23. A method for preparing a sugar-containing syrup from the stalk of a plant of the Nicotiana species, comprising:
i) removing an aqueous liquid component comprising sugar compounds from the stalk of a plant of the Nicotiana species or a portion thereof; and
ii) concentrating the aqueous liquid component to increase the specific gravity of the aqueous liquid component, which results in formation of a sugar-containing syrup suitable for use as a flavorful tobacco composition in a tobacco product.

24. The method of claim 23, wherein the removing step comprises pressing the aqueous liquid component from the stalk.

25. The method of claim 23, wherein the removing step comprises contacting the stalk of the plant or portion thereof with a liquid to draw out the aqueous liquid component.

26. The method of claim 23, wherein the concentrating step comprises heating the aqueous liquid component at atmospheric pressure.

27. The method of claim 23, wherein the concentration step comprises concentrating the aqueous liquid component to a specific gravity of about 20 to about 50 g/cm³.

28. The method of claim 23, further comprising filtering the aqueous liquid component to remove solid components.

29. The method of claim 28, wherein the filtering step comprises exposing the aqueous liquid component to an ultrafiltration membrane.

30. The method of claim 28, wherein the aqueous liquid component is filtered during the concentrating step by skimming the solid components off the surface.

31. The method of claim 23, further comprising clarifying the aqueous liquid component by adding one or more clarifying agents to the aqueous liquid component.

32. The method of claim 23, further comprising adding the sugar-containing syrup to a tobacco material or a non-tobacco plant material as a carrier for the sugar-containing syrup.

33. The method of claim 32, further comprising incorporating the tobacco material or non-tobacco plant material into a tobacco product.

34. The method of claim 33, wherein the tobacco product is in the form of a smokeless tobacco composition.

35. The method of claim 34, wherein the form of smokeless tobacco composition is selected from the group consisting of moist snuff, dry snuff, chewing tobacco, tobacco-containing gums, and dissolvable or meltable tobacco products.

36. The method of claim 33, wherein the tobacco product is in the form of a smoking article.
